(19) United States
(12) Patent Application Publication
(10) Pub. No.: US 2024/0027475 A1
(43) Pub. Date: Jan. 25, 2024

(54) POROUS UNIT WITHOUT REFLECTIVE LAYER FOR OPTICAL ANALYTE MEASUREMENTS

(71) Applicant: Radiometer Medical ApS, Brønshøj (DK)

(72) Inventors: Thomas KJAER, Brønshøj (DK); Hans Peter Blaabjerg JAKOBSEN, Brønshøj (DK); Peter Jakobsen NEILSON, Brønshøj (DK); Michael TAAGAARD, Brønshøj (DK)

(21) Appl. No.: 18/258,437

(22) PCT Filed: Dec. 20, 2021
(86) PCT No.: PCT/EP2021/086910

§ 371 Date: Jun. 20, 2023

(30) Foreign Application Priority Data
Dec. 22, 2020 (EP) 20216632.8

(51) Int. Cl. G01N 33/72; G01N 33/68; B01L 3/00

(52) U.S. Cl. CPC G01N 33/72; G01N 33/6848; B01L 3/502715; B01L 2300/069; B01L 2300/0663; B01L 2300/168; B01L 2300/161

(57) ABSTRACT

There is presented a porous unit (1) for detection of an analyte (96) in a liquid (99) by optical probing, comprising a translucent element (2) with a front side (3), and a backside (4) facing away from the front side (3), wherein the front side (3) is adapted for being contacted directly with the liquid (99), or separated from the liquid (99), such as exclusively separated from the liquid (99), by one or more layers at the front side (3) of the translucent element (2), the one or more layers (5) being adapted to be non-reflective to light reaching the one or more layers at least at one angle of incidence, such as at least at normal incidence, from the translucent element (2), and/or allow internal reflection, such as total internal reflection, at an interface, such as an external interface, of light reaching the interface from the translucent element (2), wherein the translucent element (2) comprises pores (6), wherein the pores (6) are dead end pores (6) extending from respective openings (7) fluidically connecting them with the liquid (99) at the front side (3) into the translucent element (2), wherein a cross-sectional dimension of the openings (7) of the pores (6) is dimensioned so as to prevent larger particles or debris from entering the pores (6), while allowing the analyte in the liquid (99) to enter the pores (6) via diffusion.

### FIELD OF THE INVENTION

The present invention relates to a unit for detection of an analyte in a liquid by optical probing, and more particularly a porous unit for detection of an analyte in a liquid by optical probing, and furthermore relates to a corresponding method.

### BACKGROUND OF THE INVENTION

There are many different ways of detecting a subset of molecules in a liquid. Variations are from membranes, to chemical methods and biological processes. Many known processes utilize external forces for the filtration part, which can have detrimental effect on the filtrate. Other methods utilize a multi-step process with the initial step of filtering, followed by a detection step. This can lead to complex processes.

Within the field of medical devices most traditional methods of filtering and detecting comprise large volumes of the liquid needing filtration. Detecting drugs in e.g. whole blood is often not possible and a larger volume whole blood is drawn from the patient due to the need of plasma for the analysis of the drug of interest. If the analysis is needed often the veins of the patient is punctured regularly and there is a risk of anemia for the patient.

One may use a porous unit is in relation to detection of an analyte in a patient sample. The analyte can be any of a laboratory's test parameters for blood analysis which is detectable by light, e.g. spectrophotometry. Other approaches for measuring components present in a fraction of a liquid containing particles or other debris involve the separation of a fraction from cellular components by microfiltration techniques in e.g. a microfluidic device, prior to analysis of the fraction in a dedicated measurement in the microfluidic device.

However, such filtration-based approaches have several disadvantages when used for analyzing e.g. whole blood samples. Filtration devices inherently rely on a liquid flow of at least the filtrate through the pores of the filter from a sample feed to a filtrate analysis/measurement chamber. In through-flow geometries, the retentate (in the case of whole blood, the red blood cells) gradually clogs the filtration pores. In crossflow geometries, the retentate is lead along the surface of the filtering membrane, thereby reducing but not removing the problem with clogging, especially if the system is intended for repetitive use (more than 10-100 samples). Crossflow geometry also induces friction and shear interaction between the retentate and the surface of the filtering device. Complete washout of a sample after measurement may be difficult or at least very time-consuming and unreliable, at the further risk of cross-contamination between subsequent samples. Further, additional challenges for obtaining quantitative results from the optical probing may arise in such devices, due to pressure-induced deformation of the filtration membrane resulting in a change of the optical path for probing the filtrate.

It is generally desirable during optical probing to have as high a signal-to-noise ratio as possible and/or to have as high, sensitivity as possible, and preferably as high as possible specific sensitivity.

Therefore, there is a need for an improved device and method for the detection of an analyte in a liquid with a fast and reliable response. More generally, there is a need for an improved device and method for the detection of substances in a fraction of a whole blood sample with a fast and reliable response.

### SUMMARY OF THE INVENTION

An object of the present invention is to provide an improved detection overcoming at least some of the disadvantages of known sensors, systems and or methods for detecting substances in the plasma fraction of a whole blood sample, and in particular for detecting an analyte in a liquid. Additionally, or alternatively, it is an object of the present invention to have an increased signal-to-noise ratio and/or to have an increased sensitivity, and preferably an increased specific sensitivity.

According to a first aspect, the invention provides a porous unit for detection of an analyte in a liquid, such as in whole blood, such as in a whole blood sample, by optical probing, comprising
a translucent element with a front side, and a backside facing away from the front side, wherein the front side is adapted for being
   i. contacted directly with the liquid, or
   ii. separated from the liquid, such as exclusively separated from the liquid, by one or more layers at the front side of the translucent element, the one or more layers being adapted to
      1. be non-reflective to light reaching the one or more layers at least at one angle of incidence, such as at least at normal incidence, from the translucent element, and/or
      2. allow internal reflection, such as total internal reflection, at an interface, such as an external interface, of light reaching the interface from the translucent element,
wherein the translucent element comprises pores, wherein the pores are dead end pores extending from respective openings fluidically connecting them with the liquid at the front side into the translucent element,
wherein a cross-sectional dimension of the openings of the pores is dimensioned so as to prevent larger particles or debris from entering the pores, while allowing the analyte in the liquid to enter the pores via diffusion.

A possible advantage of the present invention is that filtration is or is able to be performed by diffusion where no external energy is needed and the diffusion is also fast so that measurement on the liquid that has diffused into the pores of the porous unit can be performed shortly after the liquid has been introduced into the porous unit. Another possible advantage is that the porous unit can be kept simple, with few parts and none that needs moving or changing position during filtration and measurement. Another possible advantage is that the porous unit can be kept small in size and the volume needed for a measurement is very small compared to regular filtration devices. The porous unit could be included into other applications or devices where filtration and subsequent measurement is needed on a liquid.

Another possible advantage is that no (non-internally) reflective layer, such as a metallic reflective layer, is necessary, such as dispensing with a need for spending resources in costs during production for providing the reflective layer and/or dispensing with a risk that the reflective layer gets corrupted during use or storage. In embodiments, there is provided a porous unit wherein there is no reflective layer at the front side of the translucent element, which reflective layer is being adapted to reflect light reaching the reflective layer from the backside of the translucent element.

Another possible advantage is that it is avoided that (specular) reflection from a reflective layer interferes with measurements, such as interferes without simultaneously reaping a benefit of, e.g., evanescent waves extending beyond a front surface or front side of the porous unit.

Another possible advantage is that the invention enables yielding an increased measured light intensity, which may in turn be beneficial for lowering (relatively) the noise on individual measurements, such as improving a signal-to-noise ratio.

Another possible advantage is that the invention enables yielding an increased calibration sensitivity, which may in turn be beneficial for lowering (relatively) the noise on individual measurements, such as improving a signal-to-noise ratio, and/or improving a detection limit.

Another possible advantage is that the invention enables yielding an increased specific calibration sensitivity (where specificity relates to a comparison between certain wavelengths), which may in turn be beneficial for distinguishing a sough-after-analyte from one or more other analytes.

By ‘porous unit’ is understood a unit being in part or fully porous and/or comprising one or more porous elements. It is thus encompassed, that the porous unit can be monolithic in some embodiments and non-monolithic, e.g., comprising a sandwich-structure, in other embodiments.

The term “liquid” refers to any liquid, such as whole blood, the plasma fraction of whole blood, spinal cord liquid, urine, pleura, ascites, wastewater, a pre-prepared liquid for any kind of injection, liquids with a constituent possible to detect by spectroscopy. The liquid may be understood to have a refractive index (such a real part of the refractive index), such as at or about 416 nm or at or about 455 nm, of equal to or below 1.50, such as equal to or below 1.45, such a equal to or below 1.40, such as equal to or below 1.38, such as equal to or below 1.36.

In embodiments, the liquid is a liquid sample. The term “sample” refers to the part of the liquid that is used or needed in the analysis with the porous unit of the invention.

The term “whole blood” refers to blood composed of blood plasma, and cellular components. The plasma represents about 50%-60% of the volume, and cellular components represent about 40%-50% of the volume. The cellular components are erythrocytes (red blood cells), leucocytes (white blood cells), and thrombocytes (platelets). Preferably, the term “whole blood” refers to whole blood of a human subject, but may also refer to whole blood of an animal. Erythrocytes constitute about 90%-99% of the total number of all blood cells. They are shaped as biconcave discs of about 7 μm in diameter with a thickness of about 2 μm in an un-deformed state. The erythrocytes are highly flexible, which allows them to pass through very narrow capillaries, reducing their diameter down to about 1.5 μm. One core component of erythrocytes is hemoglobin which binds oxygen for transport to the tissues, then re-leases oxygen and binds carbon dioxide to be delivered to the lungs as waste product. Hemoglobin is responsible for the red color of the erythrocytes and therefore of the blood in total. Leucocytes make up less than about 1% of the total number of all blood cells. They have a diameter of about 6 to about 20 μm. Leucocytes participate in the body's immune system e.g. against bacterial or viral invasion. Thrombocytes are the smallest blood cells with a length of about 2 to about 4 μm and a thickness of about 0.9 to about 1.3 μm. They are cell fragments that contain enzymes and other substances important to clotting. In particular, they form a temporary platelet plug that helps to seal breaks in blood vessels.

The terms “blood plasma” or “plasma” refer to the liquid part of the blood and lymphatic liquid, which makes up about half of the volume of blood (e.g. about 50%-60% by volume). Plasma is devoid of cells. It contains all coagulation factors, in particular fibrinogen and comprises about 90%-95% water, by volume. Plasma components include electrolytes, lipid metabolism substances, markers, e.g. for infections or tumors, enzymes, substrates, proteins and further molecular components.

The term “wastewater” refers to water that has been used, as for washing, flushing, or in a manufacturing process, and so contains waste products and/or particles and is thus not suitable for drinking and food preparation.

By ‘analyte’ is understood any entity, substance or composition, and may in particular be an element, ion and/or molecule. ‘Analyte’ is understood to encompass a group of analytes, or a group of entities, substances or compositions, such as a group of entities, substances or compositions sharing one or more properties, such as chemical properties or structure or optical or physical properties.

By ‘detection of an analyte in a liquid’ may be understood both qualitatively detecting a presence (yes/no) of an analyte and quantitatively determining a concentration, such as on an ordinal, interval or ratio type scale. ‘Optical probing’ is understood as is common in the art, such as irradiating light onto at least a portion of the liquid and receiving at least a portion of light, where the received light enables deriving information about analytes (possibly) therein.

In general, when referring to optical properties (such as translucent, absorbing, internally reflective, reflective) throughout this application, it may generally be understood to be done with reference to electromagnetic radiation (or light) with wavelengths, such as at least for one wavelength, within the range from 380 nm to 750 nm, such as from 400 to 520 nm, such as 400-460 nm (or 415-420 nm), such as at or about 415 nm or at or about 416 nm or at or about 450 nm or at or about 455 nm.

The porous unit comprises a translucent element, such as a slab. The translucent element contains small, dead end pores extending from the front side, through the one or more layers (if present) into the translucent element. The porous unit needs a light source and a (light) detector arranged to optically probe the content of the pores, and to generate a corresponding signal output representative of the analyte content in a liquid.

The term “translucent” refers to a material's property of allowing light to pass through. The term “transparent” refers to the property of a material of allowing light to pass through the material without being scattered. The term “transparent” is thus considered a sub-set to the term “translucent”.

Preferably the membrane, such as the one or more layers, shows a reflectivity (such as at the interface between the translucent element and the one or more layers) of more than 25%, such as more than 30%, such as more than 35%, such as more than 40%, such as more than 50%, such as more than 75%, such as more than 90% or even more than 99% in the spectral range of detection when tested in an integrating sphere, i.e. in the spectral range from which a signal representative of the relevant plasma component is developed, such as in the range from 380 nm to 750 nm, from 400 to 525 nm, or at or about 416 nm or at or about 455 nm, e.g. for normal incidence light.

The technology applied to measure reflectance from an interface or transmittance through an interface or through a length of a (bulk) material (of light possibly being or comprising diffuse light) may be using an Integrating Sphere, such as relying on Fourier Transform Infrared (FTIR) analysis. The light hits the (possibly diffusing) sample (such as interface or a portion of bulk material) such as the interface between the translucent element and the one or more layers at a normal 90° angle to the one or more layers. The reflected and/or transmitted light is scattered when interacting with the sample. The integrating sphere is a device where scattered transmitted and/or reflected light from a diffuse sample is collected, using the highly reflective surface of the sphere wall where the light ‘bounces’ around until reaching the detector. In this way accurate results from a surface that normally would yield low reflectance or transmittance due to scattering, can be achieved.

By ‘translucent (element)’ may in general be understood an element comprising a translucent material, such as wherein said material (such as the translucent material and/or the material of the translucent element) has an attenuation coefficient so that a (optionally partially or wholly diffuse) transmission coefficient of light through the material (such as disregarding any interface effects) is at least 50% for a length through the material of 100 micrometers, such as a fraction of light not making it through a length of material is equal to or less than 50% pr. 100 micrometer, such as equal to or less than 40% pr. 100 micrometer, such as equal to or less than 20% pr. 100 micrometer, such as equal to or less than 10% pr. 100 micrometer, such as equal to or less than 5% pr. 100 micrometer, such as at a wavelength at or about 416 nm or at or about 455 nm. An advantage of this may be that it enables getting photons in to and/or out of the translucent element. The wording ‘translucent element’ may be understood and used interchangeably with ‘an element comprising translucent material’. In an embodiment, a transmission coefficient of light through the translucent element, such as from the front side to the back side in a direction orthogonal to the front side and/or the back side, such as disregarding any interface effects, is at least 10%, such as at least 25%, such as at least 50%, such as at least 75%, such as at least 90%, such as at least 95%, such as at least 99%, such as for electromagnetic radiation (or light) with wavelengths, such as at least for one wavelength, within the range from 380 nm to 750 nm, such as from 400 to 520 nm, such as 400-460 nm (or 415-420 nm), such as at or about 415 nm or at or about 416 nm or at or about 450 nm or at or about 455 nm.

The terms ‘back side’, ‘backside’ and ‘back’ are used synonymously and interchangeably.

By ‘attenuation coefficient’ may be understood Napierian attenuation coefficient u, such as wherein transmission T through a material is given as:

T=exp(−int(u(z)dz),

where ‘exp’ denotes the exponential function, ‘int’ denotes an integral (through the length of the material), z denotes a corresponding axis through the material and the corresponding coordinate).

The attenuation coefficient may be obtained as is common in the art, such as via measurement in a standard spectrophotometer, which measures the absorption through, e.g., a 1 cm cuvette. The measured absorbance, denoted by A (or Abs), is in a standard apparatus determined as A=log(I₀/I), where log is the base-10 logarithm, I₀ is the intensity before the cuvette and I the intensity after the cuvette. The measured absorbance is thus related to the Napierian attenuation coefficient as A=log(e) int(u(z)dz with e=2.71828 denoting the base number for the natural logarithm.

According to an embodiment, there is presented a porous unit wherein the translucent element is a translucent slab, such as wherein slab is understood to be monolithic.

Each of the small pores has an opening through which it can communicate with a liquid space at the front side of the translucent element. The pores thus penetrate the one or more layers (if present) to allow for liquid communication between the pores and the liquid space. The pores extend from the respective opening at the front side into the translucent element in a direction towards the backside. The pores are “dead end” meaning that the pores end within the translucent element. The pores do not continue all the way through the translucent element to the backside or to any common reservoir or recipient inside the element. The pores are only in liquid communication with the liquid space at the front side of the translucent element. Note that in some embodiments the dead end pores can be crisscrossing and at least some of the pores may thus be connected to each other forming an X-shape, a Y-shape, a V-shape, or similar interconnected shapes. Such a configuration is equally considered as dead end, since the pores are only filled from the front side and no significant net mass transport passing through the pores occurs under operation, even if they cross each other. By appropriately dimensioning the opening of the pores at the front side it is possible to prevent e.g. red blood cells of a whole blood sample or debris in the liquid on the front side of the porous unit from entering the pores, while allowing relevant components in the plasma fraction of the whole blood sample or in the liquid to enter the pores, wherein relevant components are substances present in the plasma fraction of the whole blood sample and that are to be measured/detected using the sensor. In particular, bilirubin and carbon dioxide are relevant components.

Under operation, the front side of the translucent element is contacted with a whole blood sample or a liquid. The small pores in the translucent element communicate with the whole blood sample or liquid through the openings in the front side. The pore openings are dimensioned to selectively extract a sub-sample of the plasma phase of the whole blood sample or to extract a sub-sample of the liquid including the analyte. No red blood cells can enter the pores through the openings on the front side of the translucent element. Nothing larger than the pore diameter can enter the pores which excludes e.g. any debris included in the liquid. As mentioned, the pores are dead end, only communicating with the front side of the translucent element, i.e. the sub-sample is extracted for optical probing inside the pores and after the measurement discharged again through the same openings in the front side of the translucent element. The sub-sample volume corresponds to the total internal volume of the pores. No filtration and net mass transport of any filtrate occurs through the pore containing layer—neither into any common filtrate recipient nor to any filtrate outlet. The optical detection will then be performed only on the sub-sample contained in the pores.

In an embodiment, one or more layers are present and comprises an optically absorbing layer, which optically separates the optical probing region in the translucent element from the liquid space containing the whole blood sample or the liquid. By optically separating the probing region from the liquid space, any contribution of the intact red blood cells of the whole blood sample or of the debris in the liquid to the probed signal can be effectively suppressed. The measurement is thus specific to the content of analyte in the liquid in the pores.

The small sub-sample with a representative content of the relevant components may be transferred to the pores in any suitable manner. The small dead end pores allow for a very efficient and fast extraction of the sub-sample for optical probing from a whole blood sample or a liquid through the openings in the front side by means of capillary forces and/or diffusion.

In a typical operation mode, the front side surface of the translucent element is contacted by a rinsing liquid prior to contacting the front side with a whole blood sample or liquid that is to be analyzed. Thereby, the pores are ‘primed’ with a prefill of a liquid that is compatible with the whole blood sample or the liquid, and in particular a liquid that is compatible with the plasma phase if the liquid is whole blood, such as an aqueous solution commonly used for rinse, calibration and/or quality control purposes in blood analyzers. Typical rinse liquids used for e.g. wash-out in whole blood analyzer systems may be used as such a liquid. Rinse liquids are aqueous solutions comprising K⁺, Na⁺, Cl⁻, Ca²⁺, O₂, pH, CO₂, and HCO₃⁻ in concentrations corresponding to human plasma. Non-limiting examples of suitable solutions commonly used for rinse, calibration and/or quality control purposes are given further below. When the whole blood sample or liquid is then brought in contact with the front side surface that is primed with a plasma compatible liquid, a representative sub-sample of components in the plasma phase of the whole blood sample or of the liquid is extracted and transferred in a very efficient and gentle manner by means of diffusion of the relevant components into the prefilled pore. In particular, any concentration gradient in the content of the analyte between the liquid and the reference liquid in the pores drives a diffusive transfer, thereby producing in the pores a sub-sample with an analyte concentration representative of the analyte concentration in the liquid.

According to an embodiment, there is presented a porous unit, wherein the pores are arranged to be rinsed by, such as solely by, diffusion.

In another operation mode, it may also be conceived to directly contact the front side of a dry sensor with a whole blood sample or a liquid. Further preferably in this operation mode, an inside surface of the pores is hydrophilic, thereby extracting the sub-sample from the whole blood sample or liquid at the front side of the translucent element into the pores by means of capillary forces. When operating a porous unit in this mode calibration could occur either via batch calibration as porous units produced from the same batch of porous membrane material tend to have equal sensitivity (equal light absorption when measuring on identical liquids using porous units produced from different pieces of porous membrane material from the same batch forming the translucent element). Alternatively, the pores of the translucent element can contain a calibration dye with absorption characteristics different from the analyte. The calibration dye is useful for normalizing/calibrating the optical probing signal, while being spectrally distinguishable from the substance in a plasma sample, e.g. bilirubin, to be detected/measured. Since the calibration dye will not be present in the actual liquid, the calibrant dye will diffuse out of the sensor during the measurement, mean-while the analyte diffuses into the pores of the sensor. By optically probing the pores before and after acquiring the liquid, a quantitative measure for the substance to be detected (e.g. bilirubin) may be developed by a comparison of the calibration reference and liquid substance signals.

The content of the pores can conveniently be probed optically from the back side of the translucent element, or more generally, from the side of the front surface (or front side) and/or of the one or more layers (if present) facing towards the translucent element, wherein the one or more layers (if present and including an optically absorbing layer) optically separates an optical probing region comprising the pores from the liquid contacting the front side of the translucent element, thereby preventing probing light from reaching and interacting with the liquid at the front side of the unit or the translucent element. The optical probing is thus selectively performed only on the sub-sample inside the pores. By ‘probed (optically) from the back side (of the translucent element)’ may generally be understood that incident probing light to the pores travels in a direction towards the front side from the backside (such as entering the translucent element via the backside in a direction from the backside to the front side) and light emitted from the pores to a receiving unit, such as a light detector, being emitted in a direction from the front side to the backside, such as being emitted from the back side in a direction away from the front side.

The porous unit may be connected to a light source and a light detector configured for optically probing the translucent element, the light source should be adapted to illuminate at least the pores, and the detector should be arranged to receive light emerging from the pores in response to an illumination by the light source, and the detector should also be adapted to generate a signal representative of the detected light.

Incident light is guided/directed to the optical probing region to ensure that the light traverses the pores and interacts with the sub-sample therein. Preferably, the probing light is sent into the probing region at an oblique incidence with respect to a surface normal on the plane of the surface of the front side of the translucent element and/or one or more layers (if present), to ensure that the light traverses the pores filled with the liquid to be probed, thereby ensuring a maximum of optical interaction path length.

Light emerging from the pores in response to the illumination has interacted with the sub-sample in the pores and thus carries information on the sub-sample. The emerging light and/or a signal representative of the emerging light may then be analyzed with respect to that information in order to develop a value representative of the analyte content in the whole blood sample or in the liquid. Analysis may include spectrally analyzing the emerging/detected light, and/or signal/data processing, e.g. for com-paring the obtained signal with signals obtained on calibration/reference samples, for noise filtering, for applying corrections, and for removing artefacts.

By ‘contacted directly with the liquid’ may be understood that the front side surface of the translucent element is a solid-liquid interface, such as wherein no one or more layers separate the translucent element from a volume external to the porous unit, such as the liquid. A possible advantage of this may be that no one or more layers is required, hence enabling dispensing with resources and costs for providing the one or more layers.

Additionally, or alternatively, one or more of intensity and sensitivity may be improved due to omission of a reflecting layer.

By ‘separated from the liquid by one or more layers at the front side of the translucent element’ may be understood that one or more layers, such as thin-film layers (such as a thin film layer being equal to or less than 100 micrometers thick), are present at the solid-liquid interface at the front side of the porous unit. By ‘exclusively separated’ may be understood that no other layers are separating the translucent element from the liquid.

In embodiments, the one or more layers consist of non-metallic layers. A possible advantage is that metallic reflection is avoided (where it is noted that metallic reflection may be relatively poor, e.g., with respect to internal reflection, such as internal reflection at an interface where materials on each side of the interface are non-metallic). By ‘being adapted to be non-reflective to light reaching the one or more layers at least at one angle of incidence’ may be understood that at at least at one angle of incidence (such as normal incidence), little or no light is reflected (such as a reflection coefficient being less than 0.95, such as less than 0.9, such as less than 0.8, such as less than 0.7, such as less than 0.6, such as less than 0.5, such as less than 0.4, such as less than 0.3, such as less than 0.1, such as less than 0.01) from the one or more layers when incident light is coming in a direction through, such as in a direction from, the translucent element.

By ‘being adapted to be non-reflective to light reaching the one or more layers at least at one angle of incidence’ may for example be understood that at least at one angle of incidence (such as normal incidence), a reflection coefficient is less than 0.6, such as less than 0.5, such as less than 0.4, such as less than 0.3, such as less than 0.1, such as less than 0.01, from the one or more layers when incident light (at or about 416 nm or at or about 455 nm) is coming in a direction through, such as in a direction from, the translucent element.

For example, the non-reflectivity can be due to absorption and/or transmission. The at least one angle of incidence can be normal incidence. A possible advantage of this may be that little or no reflected light from the front side can interfere with the measurements.

According to an embodiment, there is presented a porous unit wherein the front (side) of the translucent element is separated from the liquid, such as exclusively separated from the liquid, by one or more layers at the front side of the translucent element, the one or more layers being adapted to be translucent to light reaching the front side at normal incidence from the translucent element. A possible advantage of this may be that little or no reflected light from the front side can interfere with the measurements. Additionally, or alternatively, one or more of intensity and sensitivity may be improved due to omission of a reflecting layer.

According to an embodiment, there is presented a porous unit wherein the front (side) of the translucent element is separated from the liquid, such as exclusively separated from the liquid, by one or more layers at the front side of the translucent element, the one or more layers being adapted to be absorbent to light reaching the front side at normal incidence from the translucent element. A possible advantage of this may be that little or no reflected light from the front side can interfere with the measurements. Additionally, or alternatively, optical separation between the pores and the liquid outside of the pores may be achieved.

By ‘absorbent’ may be understood that more than 1%, such as more than 10%, such as more than 25%, such as more than 40%, such as more than 50%, such as more than 60%, such as more than 75%, such as more than 90%, of the incident light (at or about 416 nm or at or about 455 nm) at at least one angle of incidence (such as normal incidence) is neither reflected from the one or more layers back into the translucent element nor transmitted through the one or more layers.

According to an embodiment, there is presented a porous unit wherein the translucent element and/or the one or more layers separating, such as exclusively separating, the front side of the translucent element from the liquid is arranged for enabling internal reflection, such as total internal reflection, at the interface between on one side the translucent element and/or one or more layers and on the other side the liquid. A possible advantage may be that one or more of intensity and sensitivity may be improved due to omission of a (metallic) reflecting layer, which inhibits transmission in all directions and angles, and/or which does not allow an evanescent wave to penetrate the reflecting layer.

By ‘being adapted to allow internal reflection’ is be understood that (internal) reflection is allowed and possible at an interface between media wherein the medium comprising incident and reflected light, such as the medium wherein both incident and reflected light is travelling, is the medium of (relatively) higher refractive index compared to the medium on the opposite side of the interface, which is the medium of (relatively) lower refractive index, such as a reflection coefficient being at least 0.25, such as at least 0.4, such as at least 0.5, such as at least 0.6, such as at least 0.75, such as at least 0.90, 0.95, such as at least 0.99, e.g., at or about 416 nm or at or about 455 nm and/or normal incidence or non-normal incidence, e.g., with 45° angle with respect to normal). In embodiments, the extinction coefficient of both media (i.e., each media on each side of the interface) has an extinction coefficient (or attenuation coefficient) being sufficiently low in order for each material to qualify as translucent.

In a particularly advantageous embodiment, it is the coloring of the plasma by bilirubin that is probed optically, e.g. by using spectrally resolved absorbance measurements, or by measuring the spectrally integrated absorbance over a predetermined bandwidth within a spectral range indicative of the presence of bilirubin and/or cell-free hemoglobin in the liquid sub-sample, such as within a spectral range of wavelengths 380 nm-750 nm, such as within a spectral range of wavelengths 400 nm-520 nm, or at or about 416 nm or at or about 455 nm.

According to an embodiment, there is presented a porous unit wherein a cross-sectional dimension of the openings of the pores is 1 μm or less, such as 800 nm or less, such as 500 nm or less, such as 400 nm or less, and/or wherein a length of the pores in an axial direction along the pores is less than 100 μm and optionally larger than 5 μm, such as less than 50 μm, such as less than 30 μm, such as 25 μm.

By using pores having an opening in the plane of the front side of the translucent element with a maximum cross-sectional dimension of about 1 μm or less, or preferably in the submicron range, such as about 800 nm or less, such as about 500 nm or less, or even about 400 nm or less, any cellular components including erythrocytes, leucocytes, and thrombocytes (platelets), are prevented from entering the pores.

Further surprisingly, pores with an opening having a cross-sectional-dimension of about 500 nm or less have an increased sensitivity as compared to larger pores, such as pores having an opening with a cross-sectional dimension of about 800 nm or above, but having the same total pore volume/volume porosity.

Most preferably, the pores have a minimum opening with a respective minimum pore volume to allow for the efficient extraction of a sufficiently large sub-sample that can still be probed with an acceptable signal to noise ratio. Advantageously, the pores have an opening of about 30 nm or more, or 50 nm or more, or 100 nm or more, or about 200 nm or more.

Suitable pores may be produced e.g. from transparent polymer membranes with so-called track-etched pores, similar to those available from the company IT4IP (IT4IP s.a./avenue Jean-Etienne Lenoir 1/1348 Louvain-la-Neuve/Belgium) with the modification that the pores are closed at one end. Through-going pores in the mem-branes may be closed e.g. by laminating a backing sheet to the backside of the porous membrane, or by decelerating the ions such that the ion-bombardment tracks, and thus the pores etched following these tracks, stop within the transparent polymer membrane to form dead end pores. The membrane is typically backed by a stiff transparent element to provide adequate mechanical strength to the translucent element.

According to an embodiment, there is presented a porous element wherein the translucent element is made of a transparent polymer.

According to an embodiment, there is presented a porous element wherein the pores are track-etched in the translucent element and optionally in the one or more layers if present.

The transparent element should preferably be made of a material that does not absorb light and at the same time it should be possible to produce the dead end pores in the material e.g. by track etching the material. Material suitable for this is polyethylene terephthalate (PET or PETE) or an analogue of PET (polyethylene terephthalate pol-yester (PETP or PET-P)) or a polycarbonate (PC). The transparent element may comprise a hydrophilic coating of e.g. polyethylene glycol (PEG) to increase the diffusion into the pores. The hydrophilic coating may be chosen according to the use of the porous unit. In some use cases, the porous unit will never dry out, once it is in use and it therefore only needs to be hydrophilic at startup.

For other uses of the porous unit, it needs coating that keeps it hydrophilic permanently for allowing the porous unit to dry out and still be useable afterwards when the porous unit is re-wetted for a further use.

According to an embodiment, there is presented a porous unit wherein
a porosity of a given volume of the translucent element comprising pores is between 50% and 5% by volume, such as between 30% and 10% by volume, such as 15% by volume.

The pores create porosity in the translucent element (or in a given region of the translucent element) with a corresponding front side surface area over which the openings of the pores are distributed. The porosity may be characterized in terms of the volume of the voids created in the translucent element by the pores, i.e. the pore volume, wherein the pore volume is referred to the volume of the translucent element penetrated by the pores. This volume is here defined as the volume between the front side area over which the pores are distributed and the identical parallel area shifted into the translucent element by the maximum depth of penetration of the pores into the translucent element as seen in an axial direction perpendicular to the front side of the translucent element.

In addition thereto, the porosity may be further characterized in terms of the integrated pore volume, which is equal to the sub-sample volume that is available for optical probing. The pore volume may conveniently be expressed as an equivalent pore volume depth DELTA, which is the pore volume referred to the corresponding front side area over which the pore openings are distributed. Accordingly, the porosity of the translucent element can be converted into an equivalent pore volume depth DELTA as follows. The pores having an opening within a given front side area A have a total pore volume V. The equivalent pore volume depth is then calculated as the total pore volume divided by the given front side area: DELTA=V/A.

According to an embodiment, there is presented a porous unit wherein
an equivalent pore volume depth (DELTA) is less than 20 μm, such as less than 10 μm, such as 5 μm or less, wherein the equivalent pore volume depth (DELTA) is defined as the total volume of the pores (V) divided by the front side area (A) over which the openings of the pores are distributed.

Thereby, a small sub-sample with a representative concentration of relevant components is obtained. A small sub-sample volume is desirable to promote a fast sub-sample exchange, thereby reducing response time of the porous unit, and cycle time of measurements using the porous unit. A small sub-sample volume is further desirable in order to avoid effects of depletion of boundary layers of a plasma fraction in a whole blood sample close to the front side of the translucent element. Such depletion effect may otherwise occur in small, still standing samples, where e.g. red blood cells may obstruct an efficient diffusive exchange of relevant components from the volume of the whole blood sample towards the boundary layer at the front side of the translucent element, if the equivalent pore volume depth exceeds a critical value.

Preferably, an equivalent pore volume depth DELTA is at least 1 μm, alternatively at least 2 μm, or in the range from 3 μm to 5 μm, wherein the equivalent pore volume depth is defined as above. A larger sub-sample volume is desirable to achieve a better signal-to-noise level due to a larger sub-sample volume contributing to the optically probed information on the relevant components in the plasma.

Further according to some embodiments, a useful compromise between reducing response time, reducing cycle time, and/or avoiding depletion effects in small still standing whole blood samples or liquids on the one hand, and a required or desired signal-to-noise ratio on the other hand is found for an equivalent pore volume depth DELTA in the range from 1 μm to 20 μm, preferably in the range from 2 μm to 10 μm or at about 4 μm-5 μm.

Advantageously according to one embodiment the translucent element is supported by a translucent backing attached to the back side of the translucent element. Thereby, an enhanced mechanical stability is achieved.

According to an embodiment, there is presented a porous unit wherein a transparent backing slide of the translucent element is provided with 45°-75° angled surface (such as no 90° corners on the outside of the translucent element, the corners are “cut off” to obtain 45°-75°, such as 60°, surfaces instead), such as 60° angled surface, with respect to a front side surface, to minimize the effect of the shift in refractive index between outside air and the transparent backing slide.

Further according to one embodiment of a porous unit according to the invention, the transparent backing attached to the back side of the translucent element has such a thickness that 60° prisms (i.e. no 90° corners on the outside of the translucent element, the corners are “cut off” to obtain 60° surfaces instead) are positioned on the outside of the transparent backing for the light from the light source and to the (light) detector is having an increased angle of incidence for the light reaching the pore zone. A possible advantage of having, e.g., 60° prisms will also increase the chance of the light travelling inside the translucent element because the light is reflected at the surfaces of the backing so there will be multiple reflections before the emerging light reaches the detector.

Further according to one embodiment of a porous unit according to the invention, an inner wall surface of the pores is hydrophilic, e.g. coated with a hydrophilic coating. Thereby, an efficient capillary driven filling of dry pores with liquid is achieved. Furthermore, a hydrophilic coating prevents certain hydrophobic substances, such as hydrophobic dyes, hemoglobin, and other proteins, from depositing inside the pores that would otherwise lead to a gradual fouling of the sensor, which is difficult to wash out with an aqueous solution. Thus, an improved device for the detection of an analyte in a liquid with a fast and reliable response may be enabled.

According to an embodiment, there is presented a porous unit, wherein an inner wall surface of the pores is coated with a hydrophilic coating.

Further according to one embodiment of a porous unit according to the invention, the light source is configured for providing an obliquely incident illuminating beam from the backside of the translucent element, wherein an illumination angle is defined as the angle of the incident beam with respect to a surface normal of a reference plane defined by the front side of the translucent element. Thereby, an increased optical interaction length is achieved, thus enhancing the interaction of the incident light with the content of the pores before it leaves the probing region for detection by the (light) detector. Furthermore, penetration of probing light into the liquid through the pore openings is prevented, due to a reduced apparent cross-section of the pore openings, as well as increased scattering spreading light into the probing region rather than through the pore openings into the liquid space on the other side of the reflective layer.

The light source may in principle be any light source that transmits light in a region where the analyte in the pores absorb light in order for the system to work, but preferably the source should have a flat spectrum characteristic, i.e. the spectrum contains no peak amplitude, as a flat characteristic will give a better response. If the light source has a non-flat spectrum, i.e. the light source has a peak amplitude; a slight change in the peak may erroneously be interpreted as a change in absorption. Due to their properties with respect to size, weight, efficiency etc. light emitting diodes are often preferred. Further according to one embodiment of a sensor according to the invention, the (light) detector is configured to collect light obliquely emerging from the backside of the translucent element, wherein a detection angle is defined as the angle of the propagation of the emerging light towards the detector with respect to a surface normal of a reference plane defined by the front side of the translucent element. The detector is configured to collect light emerging in response to illumination by the light source of the optical probing arrangement. Detecting light obliquely emerging from the backside of the translucent element reduces contributions to the detected signal from light emerging from the whole blood sample and leaking back through the front side surface and the one or layers (if present) into the probing region.

The (light) detector may be a photodiode or a spectrometer that is able to detect the absorption in the entire spectrum. Alternatively, an array or diodes may be used, where each diode emits light at different wavelengths, and a photodiode is used as a detector. The diodes may be multiplexed to emit light in different intervals. The absorption is then found by comparing the lights emitted from a diode in that particular interval compared with the light detected by the photodiode.

Further according to one embodiment of a porous unit according to the invention, a plane of incidence and a plane of detection intersect at a surface normal to enclose an azimuthal angle of at least 0 degrees, and less than 180 degrees, preferably less than 160 degrees, preferably less than 130 degrees, or preferably about 90 degrees, wherein the plane of incidence is spanned by the direction of the illuminating beam and the surface normal to the reference plane, and wherein the plane of detection is spanned by the direction of the emerging light propagation towards the detector and the surface normal to the reference plane. Thereby, contributions to the detected signal of glare from partial reflections at optical interfaces prior to passing the probing region are reduced. Such glare of light that has not interacted with the sub-sample in the probing region does not comprise relevant information and is therefore detrimental to the signal-to-noise ratio.

Optical probing light may be performed by any suitable optical probing arrangement. Such optical probing arrangement may include merely directing a beam of light to the backside of the translucent element and directing the input of an optical detector to the illuminated region. The optical arrangement may include further optical elements improving coupling of the probing light into the translucent element and improving coupling of the light emerging from the translucent element into the detector input. Such optical elements may include one or more prisms and/or lens arrangements attached/glued directly to the backside of the translucent element. Preferably, the coupling optics accommodates the “reflective” nature of the optical probing, where incoming probing light and detected emerging light are kept on the same side of the front side surface of the translucent element. Further improvements may be sought in enhancing the optical interaction of the probing light with the pores, e.g. by coupling the probing light into the translucent element at a first end, forcing the light in the probing region to essentially propagate in directions parallel to the front side of the translucent element, along the front side surface of the translucent element and traversing the pores, and collecting the emerging light from another end of the translucent element, which may be transverse or opposite of the first end.

When light sources age, they might change characteristic, e.g. emit less light or drift may affect the peak amplitude. This may be compensated by using a feedback calibration process, where the detector measures the light received through the translucent, such as transparent, element in a situation where the pores in the translucent, such as transparent, element is expected to be clean, i.e. contain no molecules in the pores absorbing light. If the amplitude of the light received is smaller than expected, the feedback loop to the light source may control that the current or voltage to the light source is increased, to compensate for the degradation of the light source. Alternatively, if the light source has changed characteristics, the calculation of the actual absorption when measuring may adjust for this change of the emitted light compared to the original factory calibration.

According to an embodiment, there is presented a porous unit wherein the translucent element is provided with reflective elements arranged inside the pores, in a mouth portion thereof, adjacent to the opening at the front side of the translucent element.

The reflective elements are applied as a reflective coating on the inner wall of the pores beginning at the opening of each pore and extending into the pore. However, only a mouth portion close to the opening of the pore is covered. Providing reflective elements around the opening of the pores improves optical separation of the probing light from the liquid chamber, thereby preventing erroneous contributions to the probed signal from e.g. red blood cells in a whole blood sample in the liquid chamber. The reflecting coating may be any suitable metal coating as discussed below.

According to an embodiment, there is presented a porous unit wherein the reflective elements are provided as a reflective coating covering only a fraction of the circumference of the mouth portion of the pores in the vicinity of the opening, wherein the fraction is about 70% or less, such as 50% or less.

By only partially covering the circumference of the pores a small reflector is provided in each pore with a concavely shaped reflecting surface facing towards the inside of the pores. The partial coverage may be produced, for example, by directional deposition of a metallic layer with the front side of the translucent element inclined with respect to the direction of deposition. The openings of the pores in the plane of the front side of the translucent element act as shadow masks. The shadow masks only al-low deposition on a part of the circumferential inner wall of the pore in a mouth region thereof, i.e. close to the opening. Thereby an array of small concave mirror elements, all oriented in the same direction, may be produced.

When illuminating these small mirror elements from the concavely shaped side the resulting emerging light is directed in a preferential direction. By placing the detector in this preferential direction an improved signal-to-noise ratio is achieved as compared to other directions and as compared to embodiments without such additional small directional mirror elements.

According to some embodiments with small mirror elements, i.e. with reflective elements having directional characteristics, an increase in intensity of the emerging light by a factor of about 3 is observed, as compared to embodiments with reflective elements without directional characteristics. In addition, it has surprisingly been observed that a further increase by about 50% or more of the relevant signal occurs when using small mirror elements applied to the inner surface of the pores at a mouth portion thereof, e.g. when probing absorbance. This therefore results in a surprising overall improvement in S/N ratio by a factor of at least about 4 to 5.

Typically, the small mirror elements are symmetric with respect to a central mirror plane. Advantageously, a plane of incidence, as determined by the incident light beam, and a detection plane, as determined by the direction of detection are also arranged symmetrically with respect to this central mirror plane. According to one simplified embodiment, the plane of incidence and the plane of detection coincide and are parallel to the central mirror planes of the small mirror elements.

Advantageously according to one embodiment the reflective elements are made of metal. Such metallic coatings can be applied in a relatively cost-effective, yet well-controlled manner with adequate reflectivity.

Advantageously according to one embodiment the reflective elements are made of platinum, palladium or an alloy comprising as a principal component platinum or palladium. These materials exhibit a good reflectivity in the spectral range of the electro-magnetic spectrum (deep violet to blue) that is relevant for the detection of free hemoglobin, e.g. by absorbance probing. Furthermore, these materials are biocompatible and do not e.g. introduce artificial hemolysis. Furthermore, these materials are chemically stable and in the chemical environment of a whole blood sample.

Alternatively, according to some embodiments, the reflective elements may be made of silver or aluminum. Further advantageously according to some embodiments, the surfaces of the reflective elements facing towards the pores are encapsulated by an additional passivation layer, thereby enhancing the lifetime of the device, in particular when using silver or aluminum as a material for the reflective elements. A suitable passivation may be made of e.g. a thin layer of SiO₂ which preferably is made transparent and has to be sufficiently thin so as to not obstruct the opening of the pores. These materials may also provide a good reflectivity in the relevant spectral range and are biocompatible and chemically stable in the environment.

Advantageously according to one embodiment, the thickness of the reflective elements are between 10 nm-100 nm depending upon the used metal. Such a layer thickness allows for applying the reflective elements by an evaporation technique without clogging of the openings of the pores at the front side of the translucent element.

Advantageously according to one embodiment the detector includes a spectrophotometer and an optical probing device is configured for the spectrophotometric analysis of the light emerging from the probing region in the translucent element. This allows for resolving the spectral signature of one or more relevant components in the light emerging from the sub-sample in the probing region.

Further according to a particularly advantageous embodiment, the optical probing device is configured for measuring absorbance. Thereby a surprisingly significant signal is obtained with a relatively simple optical set-up. This allows for easy integration of the sensor with more complex analysis set-ups, such as a blood analyzer system.

Several optically active components can be found in blood, e.g. bilirubin, carbon di-oxide (CO₂), Patent Blue V, cell-free hemoglobin and methylene blue. The porous unit makes it possible to detect bilirubin and/or cell-free hemoglobin with a sensitivity high enough to be able to report natural adult bilirubin concentrations and/or cell-free hemoglobin in hemolysed samples. The dye Patent Blue V may be used in lymphangiography and sentinel node biopsy to color lymph vessels. It may also be used in dental disclosing tablets as a stain to show dental plaque on teeth. Methylene blue is used in treatment towards high methemoglobin concentrations in patients and in as treatment of some urinary tract infections.

When analyzing the resulting spectrum from the porous unit it became apparent that the absorption spectra from whole blood or plasma has a shifted, such as negative or positive baseline. The negative baseline is caused by the porous unit reflecting a higher proportion of the incoming light towards the detector when measuring on whole blood or plasma than compared to rinse. The effect can be seen at high wavelengths (600 to 700 nm) where hemoglobin does not absorb. The effect arises from the higher refractive index caused by the high protein content in plasma as compared to rinse. The effect is about 5 mAbs (where Absorbance, Abs, is an optical unit, where 1 Abs causes a damping to 10% of the original light intensity, and where mAbs refers to milli-Abs), compared to the hemoglobin having about 15 mAbs at the hemoglobin peak wavelength (416 nm). With the detector utilizing a reference determination of the light intensity from the source, it will be possible to detect total protein (mostly human serum albumin, HSA) content of the plasma fraction of a whole blood sample with a detection limit of about 1-5 g/L.

The porous unit can be used as a reading device for color producing/consuming assays. The advantage being that it is not necessary to produce plasma before the assay.

The following types of assays may be used with the porous unit:
Sandwich assays, where the receptor ligand could be bound inside the membrane channels.
Assays where one part is bound in the pores, e.g. Bromocresol Green Albumin assay, which use bromocresol green, to form a colored complex specifically with albumin. The intensity of the color, measured at 620 nm, is directly proportional to the albumin concentration in the liquid.
Enzyme activity assays as e.g. the aspartate aminotransferase (AST) activity assay kit, where the transfer of an amino group from aspartate to α-ketoglutarate results in the generation of glutamate, resulting in the production of a colorimetric (450 nm) product proportional to the AST enzymatic activity present.

The porous unit could also be used in non-medical applications such as beer brewing, wastewater analysis, food testing and in dye production. In beer brewing a precise color is desired. The porous unit could be used to determine whether or not the beer has the desired color or not by measuring on the liquid and compare the reading with a liquid of correct color. Wastewater could be analyzed for presence or absence of a constituent. In food testing, liquids such as milk, juices and other slurries, the porous unit could be used for analysis for presence or absence of a constituent or analyte. Other chemical reactors e.g. the dye industry could be using the porous unit to obtain the desired color, content or other chemical properties for their liquids.

Advantageously according to some embodiments, the porous unit or a blood analysis system comprising the porous unit further comprises a processor configured for comparing the signal generated by the detector with a predetermined calibration reference to develop a quantitative measure of the analyte level in the liquid.

Further advantageously according to some embodiments, the calibration reference is obtained on a dye-based calibration solution, such as an aqueous solution comprising tartrazine dye. Preferably, the dye-based aqueous solution is prepared from a typical rinse liquid with the addition of the calibrant dye, such as tartrazine.

According to an embodiment, there is presented a porous unit wherein the translucent element comprises, such as predominantly comprises, such as comprises 50 w/w % or more, such as consists of, material, which has an attenuation coefficient so that an, optionally partially or wholly diffuse, transmission coefficient of light through the material, such as disregarding any interface effects, is at least 50% for a length through the material of 100 micrometers, such as a fraction of light not making it through a length of material is equal to or less than 50% pr. 100 micrometer, such as equal to or less than 40% pr. 100 micrometer, such as equal to or less than 20% pr. 100 micrometer, such as equal to or less than 10% pr. 100 micrometer, such as equal to or less than 5% pr. 100 micrometer, such as at least for one wavelength within the range from 380 nm to 750 nm, such as from 400 to 520 nm, such as within the range from 400-460 nm, such as within the range from 415-420 nm, such as at or about 415 nm or at or about 416 nm or at or about 450 nm or at or about 455 nm. This may in a simple manner enable attaining the translucent properties of the translucent element.

According to an embodiment, there is presented a porous unit wherein non-reflective to light entails that at least at the one angle of incidence, such as normal incidence, a reflection coefficient is less than 0.95, such as less than 0.9, such as less than 0.8, such as less than 0.7, such as less than 0.6, such as less than 0.5, such as less than 0.4, such as less than 0.3, such as less than 0.1, such as less than 0.01, from the one or more layers when incident light is coming in a direction through, such as in a direction from, the translucent element, such as at least for one wavelength within the range from 380 nm to 750 nm, such as from 400 to 520 nm, such as within the range from 400-460 nm, such as within the range from 415-420 nm, such as at or about 415 nm or at or about 416 nm or at or about 450 nm or at or about 455 nm.

According to an embodiment, there is presented a porous unit further comprising an optical assembly comprising a light guide core, the light guide core comprising an input branch, an output branch, and a coupling interface arranged to contact the backside (4) of the translucent element opposite to the front side, such as wherein the input branch and the output branch are arranged in a common light guide plane arranged perpendicular to a front side surface. The optical assembly, optionally coupled to the back side of the porous unit, may enable performing (such as performing in an efficient, simple and/or well-controlled manner) optical measurements, such as selective optical measurements, on the fluid, such as liquid, in the pores from the back side (such as incident probing light to the pores entering in a direction towards the front side from the backside and light emitted from the pores to the light detector being emitted in a direction from the front side to the backside) of the porous unit, such as also discussed elsewhere in the present text. A possible advantage of having the optical assembly coupled, such as rigidly coupled, to the back side of the porous unit, may be that the porous unit and the optical assembly can then together form a unit or cassette, such as a sensor unit or a sensor cassette, which can be inserted and removed from a (sensor) system, such as form a consumable, which may enable in an efficient manner overcoming—by replacement—problems with wear and/or contamination of the porous unit (such as contamination of the pores), where integration, such as effective integration, with one or more peripherals, such as optical peripherals, such as light source and/or receiving unit, such as a light detector, may be enabled and/or facilitated via the optical assembly. In an embodiment, the optical assembly is as described in the application WO2021123441A1 (wherein it is possibly referred to as optical sub-assembly), which is hereby incorporated in entirety by reference, such as described in FIGS. 1-9 and the accompanying text of said application, which are hereby additionally specifically incorporated by reference. The input and output branches may be directed towards a coupling interface between the optical assembly and the translucent element, such as the backside of the translucent element.

According to an embodiment, there is presented a porous unit further comprising a housing penetrated by a flow channel defining an axial direction, the flow channel comprising a sample space and being arranged so that the porous unit with a front side defining a sensor surface for contacting the liquid, such as when the liquid is in the sample space, the sensor surface facing towards the sample space, such as wherein the pores are configured with regard to the analyte in the liquid for diffusive liquid communication with the sample space. A possible advantage of having such housing, optionally rigidly coupled to the porous unit, such as the front side of the porous unit, may be that the porous unit and the housing (and optionally furthermore the optical assembly) can then together form a unit or cassette, such as a sensor unit or a sensor cassette, which can be inserted and removed from a (sensor) system, such as form a consumable, which may enable in an efficient manner overcoming—by replacement—problems with wear and/or contamination of the porous unit (such as contamination of the pores), where integration, such as effective integration, with one or more peripherals, such as a (micro-)fluidic system (and optical peripherals in case of the optical assembly), may be enabled and/or facilitated via the optical assembly. In an embodiment, the housing (and optionally the optical assembly) is as described in the application WO2021123441A1 (wherein the optical assembly is possibly referred to as optical sub-assembly), which is hereby incorporated in entirety by reference, such as described in FIGS. 1-9 and the accompanying text of said application, which are hereby additionally specifically incorporated by reference.

In an embodiment, the porous unit with optical assembly and optionally the housing forms a cassette, such as a coherent unit which can form part of a system according to the second aspect, such as wherein the cassette can be operatively and reversibly (in an optionally non-destructive manner) connected to the remainder of the system. In a further embodiment, the cassette and the remainder of the system can be connected by a transition fit, such as a reversible friction fit. By a ‘transition fit’ is understood a fit where the parts to be held together are held securely, yet not so securely that it cannot be disassembled, such as disassembled without tools, such as disassembled by the hands a human, such as a normal person. In a further embodiment, different parts of the equipment are kept together by a mechanical locking member, such as one or more or all of: A pin (such as a split pin, or a spring pin), a click-lock (such as a lock wherein a spring loaded engagement member positioned on one part engages with a cavity or edge on another part upon assembly, so that the spring force has to be overcome before disassembly), a detent ball, a hand-operable screw, such as a tommy screw, or a wing screw. It may be understood that any of the mechanical locking members may serve to retain the parts together, but also that any of the mechanical locking members may optionally be overcome or removed without tools, such as by the hands of a human, such as a normal person.

According to a second aspect of the invention, there is presented a system comprising the porous unit according to the first aspect, and further comprising
One or more light sources, wherein the one or more light sources is adapted to illuminate at least the pores in the translucent element, and/or
a light detector, wherein the light detector is arranged to receive light (21) emerging from the pores in response to an illumination (11) by one or more light sources, such as one or more light sources, and wherein the light detector is adapted to generate a signal representative of the received light.

By ‘one or more light sources being adapted to illuminate at least the pores in the translucent element’ is understood any light source, such as any light source capable of providing sufficient light (or more particularly sufficient spectral flux within relevant wavelength ranges or enabling optically probing the analyte). The one or more light sources may comprise, e.g., an incandescent light source (such as a tungsten lamp), a fluorescent light source (such as a mercury vapour lamp), an LED light source or a LASER light source (such as an argon-ion gas laser).

‘A light detector’ is understood as is common in the art, such as an electrically operated light detector, such as outputting a signal electrically and/or digitally. It may furthermore be understood that ‘light detector’ may comprise or encompass a plurality of (sub-)light detectors. A ‘detector’ is generally understood as a ‘light detector’ and the terms ‘detector’ and ‘light detector’ are used synonymously and interchangeably.

In an embodiment, the system comprises the porous unit comprising an optical assembly and optionally the housing, such as said porous unit being a cassette being operatively and reversibly connectable to the remainder of the system.

In an embodiment there is presented a system for analyzing a liquid comprising
a liquid chamber with inlet and outlet ports for feeding and discharging the liquid,
a first detector (or detecting unit) adapted to provide a first signal representative of a level of an analyte in the liquid, and
one or more further detectors (or detecting units), each further detector (or detecting unit) being adapted to provide a respective further signal representative of the analyte of the liquid,
wherein the first and further detectors (or detecting units) are operable to obtain the first and the one or more further signals from the same liquid,
wherein the first detector (or detecting unit) is configured as, such as comprising, a porous unit for the optical detection of the analyte according to the first aspect.

As already discussed above, by this design it is achieved that the pores can be filled from the front side with a sub-sample comprising relevant components of the plasma in representative amounts, merely by contacting the front side surface of the porous unit with a liquid, and that the sub-sample thus extracted can conveniently be optically probed. Relevant components could be substances that are present in the plasma phase of a whole blood sample and that are to be measured/detected using the sensor. A representative sub-sample of the plasma phase may be extracted from the whole blood sample and transferred into the pores by means of diffusion and/or capillary forces. As also discussed above, the pores are preferably prefilled with a liquid that is compatible with the plasma phase, such as an aqueous solution commonly used for rinse, calibration and/or quality control purposes in blood analyzers. Non-limiting examples of suitable solutions are given further below. Priming the pores with such a known liquid allows for extracting a sub-sample representative of the relevant components in the plasma into the pores by diffusion alone.

Advantageously according to an aspect of the invention, a method of optically detecting an analyte such as bilirubin and/or cell-free hemoglobin in a liquid is provided as detailed in the following. The method at least achieves the same advantages as discussed above with respect to respective embodiments of a porous unit for detecting an analyte, or of a system comprising such a porous unit.

According to an embodiment there is presented a system, wherein the system, such as said system being a blood gas analyser, is further arranged for measuring a concentration in the liquid sample of one or more or all of:
Carbon dioxide, such as CO₂,
Oxygen, such as O₂, and
pH.

An advantage of having such (blood gas analyzer) apparatus may be that it enables providing further relevant liquid (blood) sample parameters, such as wherein—via the output—a user (even a non-specialized) user may be informed, e.g., if one or more analytes may be associated with a (too) high cell-free hemoglobin interference criticality, such as wherein retesting may be necessary. An advantage may for example be, that it provides a relevant solution for point-of-care testing, where one or more or all of fast response times, relevant output to non-specialized users and a plurality of parameters may be particularly relevant. According to an embodiment there is presented a system wherein the system is arranged for optically probing the liquid disposed inside the pores from the side of the front side facing the back side. A possible advantage may be that it enables avoiding that light has to traverse liquid outside of the pores (such as in front of the front side) on its way to and/or from the pores, which could have led to a contribution to (such as contamination of) an optical probing signal from constituents in the liquid outside of the pores (where it is noted that the pores may in themselves be beneficial for effectively filtering the liquid for the purpose of enabling obtaining a signal only from constituents small enough to enter the pores).

According to an embodiment there is presented a system comprising both one or more light sources, such as the one or more light sources, and at least a light detector, such as the light detector, and wherein each of the one or more light sources and the light detector is placed on the side of the front side facing the back side, such as outside of the translucent element on the same side of the front side as the backside. This may be advantageous for facilitating a simple and/or efficient system, such as for optically probing the liquid disposed inside the pores from the side of the front side facing the back side.

According to an embodiment there is presented a system wherein
the one or more light sources is adapted to illuminate at least the pores in the translucent element, from the side of the front side facing the back side, and/or
the light detector is arranged to receive light emerging from the pores, such as emitted in response to an illumination by one or more light sources, such as the one or more light sources, and wherein the light detector is adapted to generate a signal representative of the received light, which has been emitted, such as primarily emitted, from the pores in a direction away from the front side in a direction facing the back side.

This may be advantageous for facilitating a simple and/or efficient system, such as for optically probing the liquid disposed inside the pores from the side of the front side facing the back side.

According to an embodiment there is presented a system wherein
the one or more light sources is adapted to illuminate at least the pores in the translucent element, such as from the side of the front side facing the back side, wherein light from the one or more light sources reaching the pores need not have traversed a volume being fluidically connected with the pores and being outside of the translucent element, such as on the side of the front side opposite the back side, and/or

the light detector is arranged to receive light emerging from the pores, such as emitted in response to an illumination by one or more light sources, such as the one or more light sources, and wherein the light detector is adapted to generate a signal representative of the received light, wherein light emitted from the pores and reaching the light detector need not have traversed a volume being fluidically connected with the pores and being outside of the translucent element, such as on the side of the front side opposite the back side. A possible advantage may be that by avoiding that light has to traverse liquid outside of the pores (such as in front of the front side) on its way to and/or from the pores, a contribution to (such as contamination of) an optical probing signal from constituents in the liquid outside of the pores may be reduced, minimized or eliminated (where it is noted that the pores may in themselves be beneficial for effectively filtering the liquid for the purpose of enabling obtaining a signal only from constituents small enough to enter the pores).

According to an embodiment, there is presented a system wherein the system is configured for measuring (optionally spectrally resolved) absorbance, such as absorbance of a liquid in the pores. An advantage of this may be that it enables in a simple way obtaining information, such as information of concentration, of an analyte in the liquid in the pores.

According to a third aspect of the invention, there is presented a method for optically detecting an analyte in a liquid, such as in whole blood, such as in a whole blood sample, comprising
providing a porous unit according to the first aspect such as a system according to the second aspect,
contacting the front side of the porous unit with the liquid,
optically probing the liquid disposed inside the pores from the side of the front side facing the back side (such as wherein incident probing light reaches the pores by traveling in a direction parallel with or towards the front side of the translucent element in a direction from the backside to the front side, such that a vector defining a direction of the incident light does not have a component being parallel with a direction from the front side to the backside), and,
based on the result of the optical probing, establishing an analyte concentration of the liquid.

According to some embodiments, a method of optically detecting an analyte in a liquid comprises the steps of providing a porous unit as disclosed above; contacting the porous unit with a reference liquid so as to fill the pores with the reference liquid; contacting the front side of the porous unit with a liquid; waiting for a diffusion time to allow for diffusion of the analyte in the liquid into the pores to stabilize; optically probing the liquid inside the pores; and, based on the result of the optical probing, establishing an analyte level of the liquid. Preferably, the reference liquid is an aqueous solution that is compatible with the liquid, and in particular with the fraction thereof that may enter the pores, such as a liquid for rinse, calibration and/or quality control. In some embodiments, it may be conceived to omit the step of contacting the front side of the unit with a reference liquid prior to introducing the liquid. However, including the step allows for a purely diffusive sub-sample extraction, which is very efficient and leads to a surprisingly fast detection response and surprisingly short cycle time for the measurement. Most advantageously, an analyte is detected optically in the pores by the color change due to the presence of the analyte in representative amounts in the extracted sub-sample.

Advantageously according to some embodiments, optical probing comprises illuminating the translucent element with probing light from the backside and performing a spectrophotometric analysis of the light emerging from the backside of the translucent element as an optical response to the probing light.

Advantageously according to some embodiments, optical probing is measuring the absorbance.

Advantageously according to some embodiments, the method further comprises the step of comparing the optical response with a predetermined calibration reference to develop a quantitative measure of the analyte level in the liquid.

Further advantageously according to some embodiments of the method, the calibration reference is obtained on a dye-based calibration solution, such as an aqueous solution comprising tartrazine dye. Preferably, the dye-based aqueous solution is prepared from a typical rinse liquid with the addition of the calibrant dye, such as tartrazine.

In an embodiment there is presented a method wherein the analyte is
cell-free hemoglobin,
bilirubin, and/or
total protein content.

In an embodiment there is presented a method wherein the liquid is a whole blood sample or wherein the liquid is a plasma phase of a whole blood sample.

In an embodiment there is presented a method, further comprising:
contacting the porous unit with a reference liquid so as to fill the pores, such as so as to fill the pores by diffusion, with the reference liquid, and/or
waiting for a diffusion time to allow for diffusion of the analyte in the liquid into the pores to stabilize.

In the context of point-of-care measurement systems (in the art also referred to as ‘bedsite’ systems) and laboratory environments alike, blood gas analysis is oftentimes undertaken by users, such as nurses, who may not be users trained in use of, e.g., blood gas analyzers.

According to another aspect of the invention, there is presented use of a use of a system according to the second aspect of the invention for point-of-care (POC) analysis of a liquid, such as whole blood, such as in a whole blood sample.

POC measurement is also referred to as ‘bed site’ measurement in the art. In the present context, the term ‘point-of-care measurement’ should be understood to mean measurements which are carried out in close proximity to a patient, i.e. measurements that are not carried out in a laboratory. Thus, according to this embodiment, the user of the system, such as the system being a blood gas analyzer, performs measurement of a whole blood sample in a handheld blood sample container in the proximity of the patient, from whom the blood sample is taken, e.g. in the hospital room or ward accommodating the patient's bed, or in a nearby room of the same hospital department. In such use, the level of expertise of the user oftentimes varies from novice to experienced, and the capability of the blood gas analyzer to automatically output instructions matching each individual user's skills on the basis of sensor input is thus particularly beneficial in such environments.

According to an alternative invention, there is presented a porous unit for detection of an analyte in a liquid by optical probing, comprising
a translucent element with a front side, and a backside facing away from the front side, wherein the front side is adapted for being
   i. contacted directly with the liquid, or
   ii. separated from the liquid, such as exclusively separated from the liquid, by one or more non-metallic layers at the front side of the translucent element,
wherein the translucent element comprises pores, wherein the pores are dead end pores extending from respective openings fluidically connecting them with the liquid at the front side into the translucent element,
wherein a cross-sectional dimension of the openings of the pores is dimensioned so as to prevent larger particles or debris from entering the pores, while allowing the analyte in the liquid to enter the pores via diffusion.

A possible advantage (of this alternative invention) is that metallic reflection is avoided (where it is noted that metallic reflection may be relatively poor, e.g., with respect to internal reflection, such as internal reflection at an interface where materials on each side of the interface are non-metallic).

The first, second and third aspect of the present invention may each be combined with any of the other aspects. These and other aspects of the invention will be apparent from and elucidated with reference to the embodiments described hereinafter.

### BRIEF DESCRIPTION OF THE FIGURES

The porous unit, system and method according to the invention will now be described in more detail with regard to the accompanying figures. The figures show one way of implementing the present invention and is not to be construed as being limiting to other possible embodiments falling within the scope of the attached claim set.

Preferred embodiments of the invention will be described in more detail in connection with the appended drawings, which show in

FIG. 1 schematically, a porous unit device according to one embodiment, under operational conditions,

FIG. 2 schematically, a cross-sectional detail of a pore, with an additional reflecting element, according to one embodiment;

FIGS. 3a/b schematically, two cross-sectional side views of a detail of a pore, with an additional reflecting element, according to a further embodiment;

FIG. 4 schematically, a cross-sectional side view of a measurement cell;

FIG. 5 a top elevational view of the measurement cell of FIG. 4;

FIGS. 6a/b schematically, two cross-sectional side views of a measurement cell with prism-like outside of the transparent backing, according to a further embodiment;

FIG. 7 a top elevational view of the measurement cell of FIG. 6a;

FIG. 8 a graph showing examples of response of bilirubin in plasma;

FIG. 9 a graph showing IR spectra of CO₂ and H₂O (retrieved from http://www.randombio.com/co2.html on Nov. 8, 2016);

FIG. 10 a graph showing an example of using a dye (Tartrazine) as a calibration and quality control reference for spectrophotometric measurements;

FIG. 11 a graph showing examples of response to different concentrations of protein (HSA) in human whole blood; and

FIG. 12 graphs showing temporally resolved signals for a porous unit.

### DETAILED DISCLOSURE OF THE INVENTION

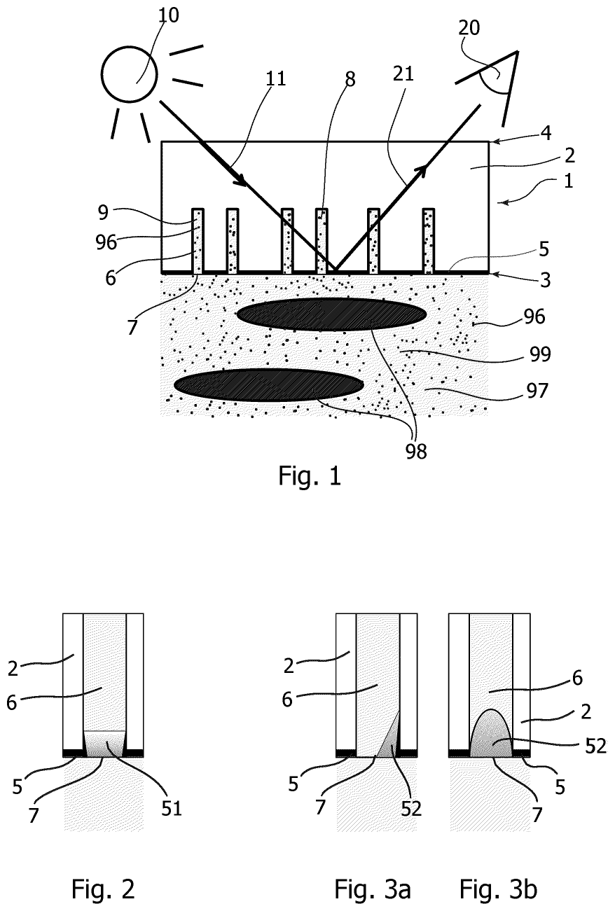

FIG. 1 shows schematically, a cross-sectional view of a porous unit 1 according to one embodiment. The porous unit 1 comprises a translucent element 2, with a front side 3 and a back side 4. The front side 3 is provided with a one or more layers 5 enabling internal reflection (in a possible embodiment there would be no one or more layers and in an alternative embodiment, there would be one or more translucent layers and in yet another alternative embodiment, there would be one or more absorbing layers). The translucent element 2 further comprises dead end pores 6 extending from an opening 7 at the front side 3 through the one or more layers 5 into the bulk of the translucent element 2, where they terminate. While shown like that in the schematic drawing of FIG. 1, the pores do not have to be perpendicular to the front side 3 or parallel to each other. Under operation, the front side 3 of the porous unit with pore openings 7 is contacted with a liquid 99. The liquid may have a cellular fraction or particular fraction comprising red blood cells or particles 98, and a plasma fraction/liquid fraction 97 with relevant components to be detected, here the analyte 96. A cross-sectional dimension of the openings 7 of the pores 6 is dimensioned so as to prevent red blood cells or particles 98 from entering the pores 6, while allowing the analyte 96 to enter the pores 6.

The pores 6 may be pre-filled with a rinse solution 8 that is compatible with the liquid 99, and in particular with the liquid fraction 97. When the liquid 99 contacts the front side 3 of the porous unit 1 with the pre-filled pores 6, a diffusive transfer of the analyte 96 into the pores 6 occurs, thereby establishing a sub-sample 9 inside the pores 6 with a concentration of the analyte 96 that is representative of the concentration of the analyte 96 in the liquid 99.

The rinse solution 8 used for pre-filling the pores 6 may be any aqueous solution compatible with the liquid 99. Suitable rinse solutions include those commonly used for rinse, calibration, and/or quality control purposes in blood parameter analyzers. Such solution compositions typically include organic buffers, inorganic salts, surfactant, preservatives, anti-coagulant, enzyme, colorant and sometimes metabolites. Optical detection is performed from the backside using an optical probing arrangement with a light source 10 and a detector 20. The light source 10 illuminates a probing volume in the porous portion of the translucent element 2 from the side of the one or more layers 5 facing away from the liquid 99. The probing light 11 is an obliquely incident beam interacting with the sub-sample 9 in the pores 6. Emerging light 21 is detected by the detector 20 also arranged to view the probing region at an oblique angle. The detector 20 generates a signal representative of the emerging light, and in particular contains information on the concentration of the analyte 96, due to the interaction with the sub-sample 9 in the pores 6. Processing the generated signal allows developing a level of the analyte in the liquid. Using calibration, the level of the analyte in the liquid may be quantitative. The optical probing technique used for all measurements in the examples below uses spectrally resolved absorbance measurements in the visible range of the electromagnetic spectrum, e.g. with wavelengths in the range between about 380 nm and 750 nm, between about 400 nm and 520 nm, or at about 455 nm.

A measurement cycle is concluded by washing out the liquid with a rinse solution, such as the rinse solution 8 used for pre-filling the pores 6. Thereby, the sensor device is re-initialized and ready for receiving the next liquid.

FIG. 2 shows a detail of a porous unit according to a further embodiment. A single pore 6 in the translucent element 2 is shown schematically. The pore 6 comprises a reflective element in the form of a reflective collar 51 produced by a deposition of reflective material into a mouth portion at the opening 7 of the pore 6.

FIG. 3a and FIG. 3b show two cross-sectional views of a detail of a porous unit according to yet a further embodiment. Again, a single pore 6 in the translucent element 2 is shown schematically. The pore 6 comprises a reflective element in the form of a small mirror element 52 produced by a directional deposition of reflective material into the mouth portion at the opening 7 of the pore 6, wherein the mirror only covers a fraction of the circumference of the opening/mouth portion as indicated in the two views of FIGS. 3a and 3b. The small mirror element 52 is concave as seen from the inside of the pore. By producing the small mirror elements with directional evaporation of a suitable reflective material, preferably metal, onto an inclined porous translucent element 2 (optionally followed by etching or grinding of a reflective layer on the front side surface of the translucent element), all mirror elements 52 are formed at the same time and pointing in the same direction. Thereby, a preferential direction of the emerging light 21 is achieved when probing light 11 is incident from the concave side of the small mirror elements 52. Consequently, the signal-to-noise ratio of a signal generated from light emerging in the preferential direction is improved considerably.

All examples given below have been measured using a sensor configuration with additional small mirror elements as obtained by a directional sputter evaporation of Pd onto the front side of a translucent polymer element 2 with a direction of evaporation at an angle of inclination of 25 degrees with respect to the surface normal on the front side 3, until one or more layers 5 with a thickness of 30 nm on the front side 3 of the translucent element 2 is obtained. The translucent element 2 is made of a translucent, preferably transparent, polymer material and has track-etched dead end pores 6 with an essentially circular cross-section. The pores have an opening 7 with a diameter of 400 nm and a depth of 25 μm distributed with a porosity of 15% by volume. Together, the pores distributed over a given front side surface area A have a total volume V and have an equivalent pore volume depth DELTA=V/A. For the above specified liquid used for measurements in the examples given below, the equivalent pore volume depth DELTA is about 4 μm.

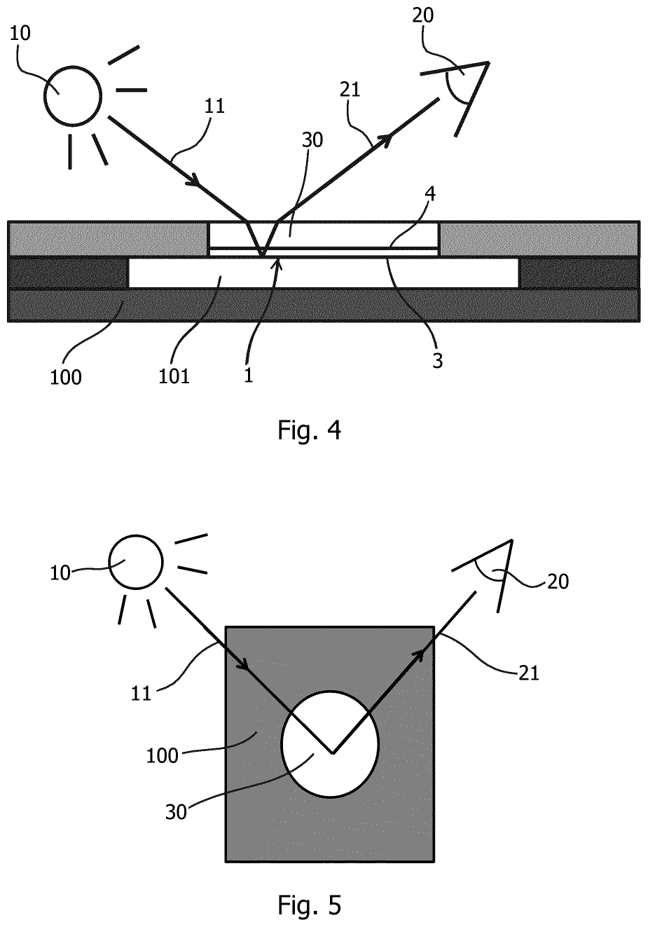

FIG. 4 and FIG. 5 show schematically a measurement cell 100 comprising a porous unit 1 with its front side 3 facing into a liquid volume 101 inside the measurement cell 100, such as wherein the measurement cell 100 is a housing and wherein the liquid volume 101 is a sample space. The liquid volume communicates with liquid input and output ports (not shown) for feeding and discharging liquids and for performing priming, rinsing, and wash-out steps. The back side of the porous unit is mechanically stabilized by a transparent backing slide 30, which also acts as a window for optical access to the probing region from the back side 4 of the porous unit 1. Optical probing is performed using an arrangement with a light source 10 and a detector 20 as described above with reference to FIG. 1, wherein the probing beam and the direction of detection are inclined with respective angles to a surface normal on the plane of the front side 3 of the porous unit 1. Furthermore, as best seen in FIG. 5, the planes of incident probing light 11 and of detection 21 preferably intersect each other with an angle of less than 180 degrees to avoid glare effects, and preferably at a pointed angle of about 90 degrees or below. In the measurements of the examples given below, the planes of incident probing light 11 and of emerging light 21 are arranged symmetrically with respect to a direction parallel to the symmetry planes of the small mirror elements 52.

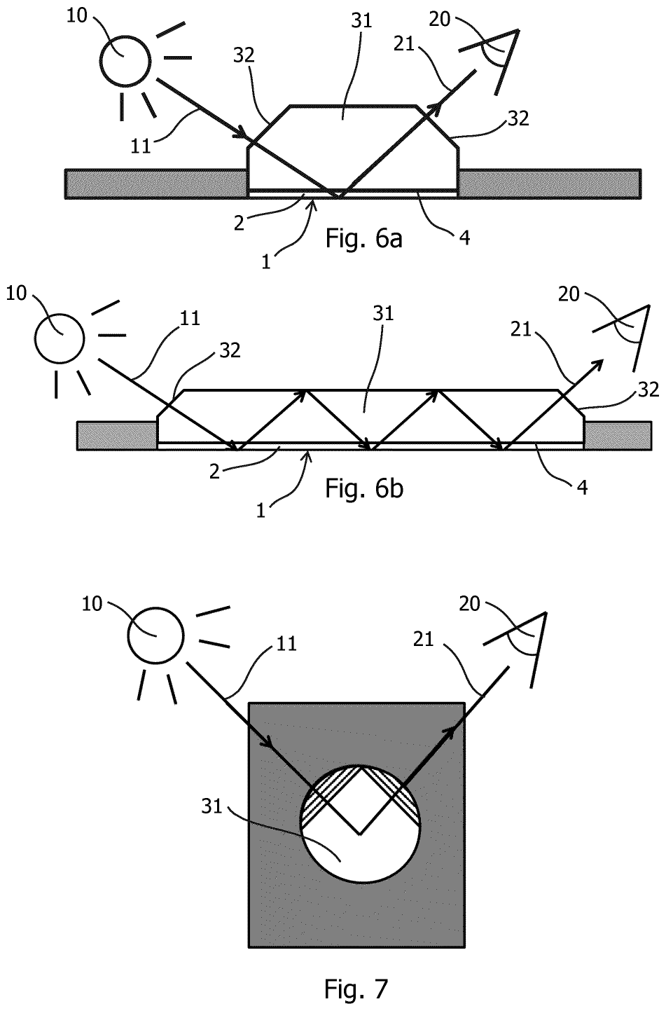

FIG. 6a, 6b and FIG. 7 show schematically a transparent backing slide 31 in direct contact with the back side 4 of the translucent element 2 of the porous unit 1. When incident probing light 11 enters the back slide 4 of the translucent element 2 with the surface at 60° prism 32, the shift in refractive index between air and polymer does not affect the incident probing light 11 and the light enter the pores 6 (not seen) of the translucent element 2 without change of the angle of the light and the emerging light 21 reaches the detector 20. FIG. 6b shows that the incident probing light 11 may be reflected several times in the transparent backing slide 31 before the emerging light 21 reaches the detector 20. Furthermore, as best seen in FIG. 7, the planes of incident probing light 11 and the emerging light 21 preferably intersect each other with an angle of less than 180 degrees to avoid glare effects, and preferably at a pointed angle of about 90 degrees or below and the prisms 32 does not affect the incident probing light 11, nor the emerging light 21.

In FIGS. 1, 4, 5, 6a, 6b and 7 the pores are probed optically from the back side 4 of the translucent element 2, i.e., incident probing light 11 to the pores 6 travels in a direction towards the front side 3 from the backside 4, i.e., entering the translucent element 2 via the backside 4 in a direction from the backside 4 to the front side 3 and light 21 emitted from the pores 6 to a receiving unit, such as a light detector 20, being emitted in a direction from the front side to the backside, i.e., from the back side in a direction away from the front side.

In FIGS. 1, 4, 5, 6a, 6b and 7 the incident and emitted light is depicted as propagating in air or empty space, but in embodiments, said incident and emitted light could be propagating in an optical assembly comprising a light guide core, the light guide core comprising an input branch, an output branch, and a coupling interface arranged to contact the backside 4 of the translucent element 2 opposite to the front side 3, such as wherein the input branch and the output branch are arranged in a common light guide plane arranged perpendicular to a front side surface.

### Examples

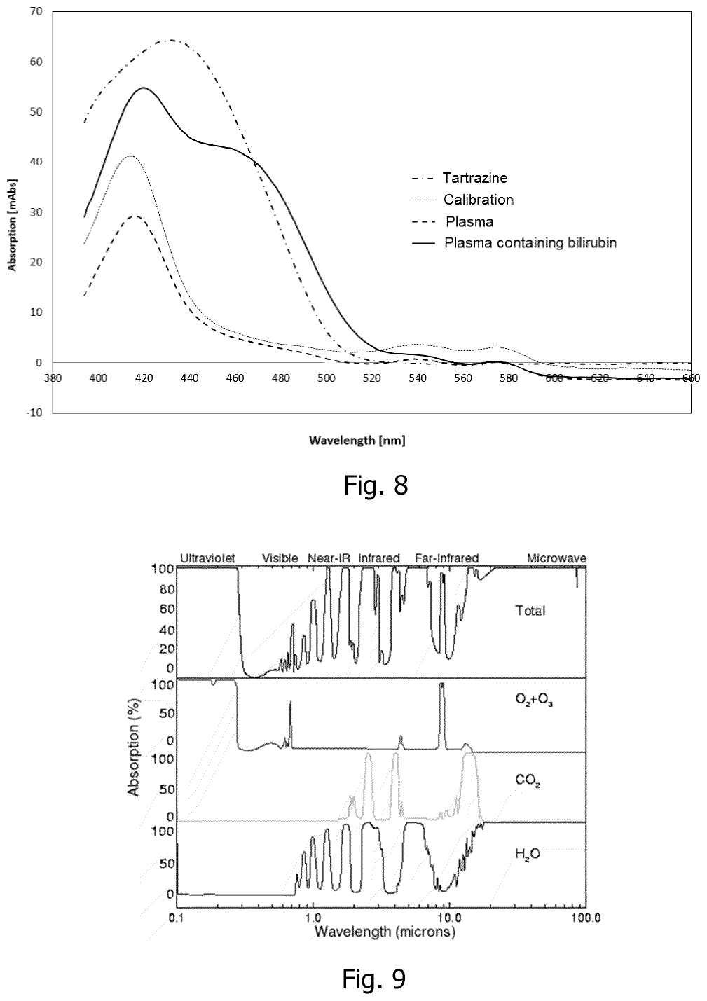

Referring to FIGS. 8-11 in the following, data from test run measurements are given as examples illustrating different aspects of the performance of a porous mirror, which corresponds to a porous unit comprising a reflective palladium layer at the front side of the translucent element (which in the examples is a slab), the reflective palladium layer being adapted to reflect light reaching the reflective palladium layer from the backside of the translucent element, wherein the data from the porous mirror are presented as examples useful for understanding the porous unit according to embodiments of the invention.

The porous mirror used for the experiments of these examples were produced from a transparent PETP-membrane, with a total thickness 49 μm that is provided with single-sided track-etched, linear pores. The pores have a pore depth of 25 μm and a pore diameter of 0.4 μm with a hydrophilic PVP treatment. The areal pore density is 1.2E8/cm². The pores are thus dead end with an opening at one side of the PETP-membrane, ending essentially half way into the PETP-membrane acting as the translucent slab. The porous side of the membrane (translucent slab) is sputter coated with Palladium at an angle of 25 degrees and with an approximate layer thickness of 30 nm. This gives a metal coating on the porous front side of the membrane (translucent slab) and a small coating on one side of the inside of the pores thus forming small concave mirrors in a mouth portion of the pores adjacent to their opening towards the front side. The sputtered porous PETP-membrane is laminated to a custom build cuvette using a double sided adhesive tape so that the concave side of the small mirrors in the pores is pointing halfway between light guides from the light source and from a spectrometer input. A drop of approximately 10 μL of silicon rubber is pipetted onto the membrane and a cover glass is then fixed to the backside of the membrane as a mechanical backing of the sensor membrane (translucent slab). The porous mirror is mounted in a test bench for automatic handling of liquids, time intervals and data sampling. Data acquisition last approximately 3 s and is delayed until 14 s after liquid acquisition.

The test bench is equipped with two light emitting diodes (a purple and a ‘white’ LED) as light source, and with a mini-spectrometer as a detector. The standard slit in the mini-spectrometer has been replaced with a 125 μm slit in order to increase light and sensitivity.

As the measurement is a reflection measurement, the light source and detector are both placed on the back side (none porous side) of the porous membrane. The porous metal coated side of the membrane is positioned on the inside of the measuring chamber and the mirror and the pores are thus directly exposed to the liquids in the chamber. Light from the two light diodes are led through a common fiber light guide, which has a lens at the end for collimating the light to a small spot of the porous mirror membrane (approximately 2 mm by 2 mm). Referring to a Cartesian coordinate °, the plane of the membrane (front side of the translucent slab) may be defined as the ZX-plane of the coordinate system. The light enters the membrane outer surface (back side of the translucent slab) at a 45° angle with respect to the Y-axis, i.e. the surface normal to the ZX-plane (and in the YZ-plane of the coordinate system). The detector is positioned with a polar angle of 60° with respect to the Y-axis, and turned with respect to the YZ-plane by an azimuthal angle of 90° with respect to the plane of incidence of the light source (e.g. in the YX-plane). The relatively high angles of light incidence and detection direction with respect to the Y-axis results in improved detection sensitivity for hemoglobin, since the collected light has traveled through a greater length of the sub-sample in the pores.

Liquids are prepared by spiking a whole blood sample with bilirubin. The interference solutions based on plasma are prepared by spiking the plasma with interferents to the specified values. Plasma is produced by centrifugation in 15 min. at 1500 G. As reference, the absorbance spectra of centrifugation derived plasma from all whole blood samples tested are also measured on a Perkin Elmer Lambda 19 UV-Vis spectrometer.

Spectral FIG. 8 shows spectrally resolved absorbance data for two fluids, one with plasma containing bilirubin and on with only plasma. At a wavelength of around 455 nm a pronounced peak is observed wherein the absorbance maximum for the different fluids evidently scales linearly according to their content in bilirubin.

Spectral FIG. 9 shows spectrally resolved infra-red data of carbon dioxide (CO₂) and water (H₂O). The non-overlaying peaks from CO₂ compared to water indicates that the CO₂ content can be determined using a porous mirror of the invention in a CO₂ containing fluid, even if water is present in the fluid.

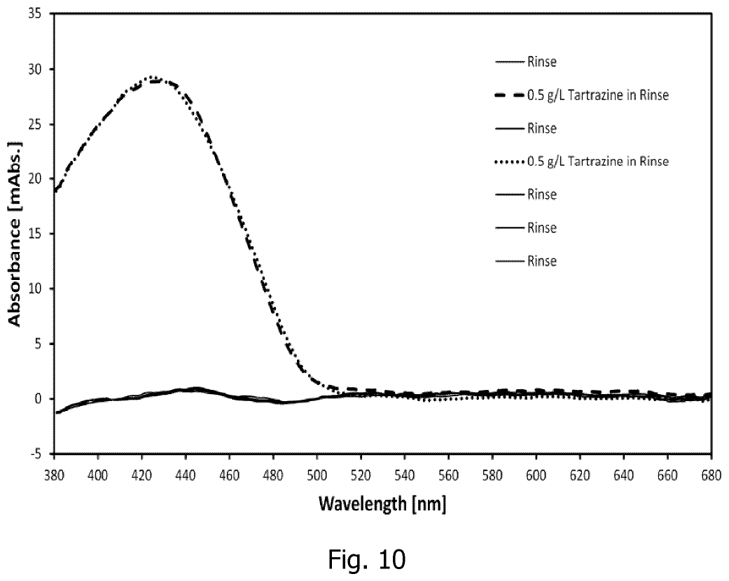

Spectral FIG. 10 shows an example with a series of spectrally resolved absorbance data obtained on a dye-based calibration solution and, for comparison, on a rinse solution. The spectra where obtained in successive cycles immediately after each other. The dye-based calibration solution is a rinse solution with an addition of 0.5 g tartrazine per 1 L rinse. The sequence of measured solutions is as follows: First a rinse solution, then a dye-based calibration solution, then again a rinse solution, again the same dye-based solution and a sequence of three consecutive measurements all performed on rinse solution. All spectra are plotted on the same scale and on top of each other. The experiment shows again a very good stability and reproducibility of the obtained results. Yet more important, the data shows a surprisingly clear separation of the two dye-based solution spectra coinciding on top of each other, and all five rinse solution spectra also coinciding on top of each other. Note that the optical data are all probed in the probing volume of the porous mirror. This indicates a very efficient and complete diffusive exchange for extraction and washout of the sub-sample in the pores also when using a dye-based spectrophotometric calibration solution, such as the above-mentioned tartrazine dyed rinse solution.

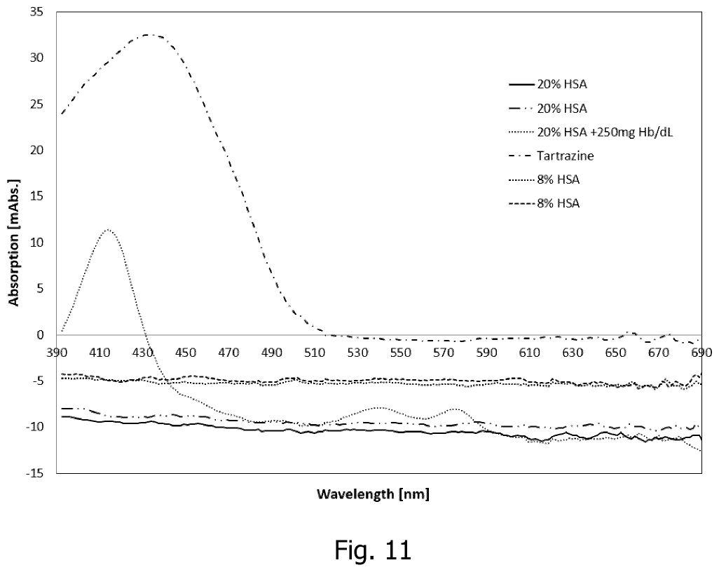

Spectral FIG. 11 shows spectrally resolved absorbance data of the negative baseline caused by the higher refractive index by the high protein content in plasma as compared to rinse. The porous mirror reflects a higher proportion of the incoming light towards the detector when measuring on whole blood or plasma than compared to rinse. The effect is seen at high wavelengths (600 to 700 nm) where hemoglobin in the whole blood does not absorb. The effect is about 5 mAbs, compared to the hemoglobin having about 10-15 mAbs at the hemoglobin peak wavelength (416 nm). It will be possible to detect the content of protein (HSA) of whole blood samples with a detection limit of about 1-5 g/L. Two different HSA concentrations (20% and 8%) are measured, the higher concentration is also measured with free (i.e. hemoglobin outside of red blood cells) in the liquid. The presence of hemoglobin in the liquid only affects the part of the spectra below 600 nm. Above 600 nm the HSA content is the main influence on the spectra, the more negative baseline the higher protein content in the whole blood sample.

While the device and method of the invention has been discussed specifically with reference to the detection of bilirubin and/or cell-free hemoglobin, according to a broader aspect, the devices and methods discussed herein are equally applicable to the detection of other optically active substances in the plasma fraction of a whole blood sample or in a liquid, wherein “the term optically active” refers to substances, that can be detected directly by a spectroscopic optical probing technique. Such substances may include, but are not limited to metabolic substances, pharmaceutical substances, drugs, or vitamins.

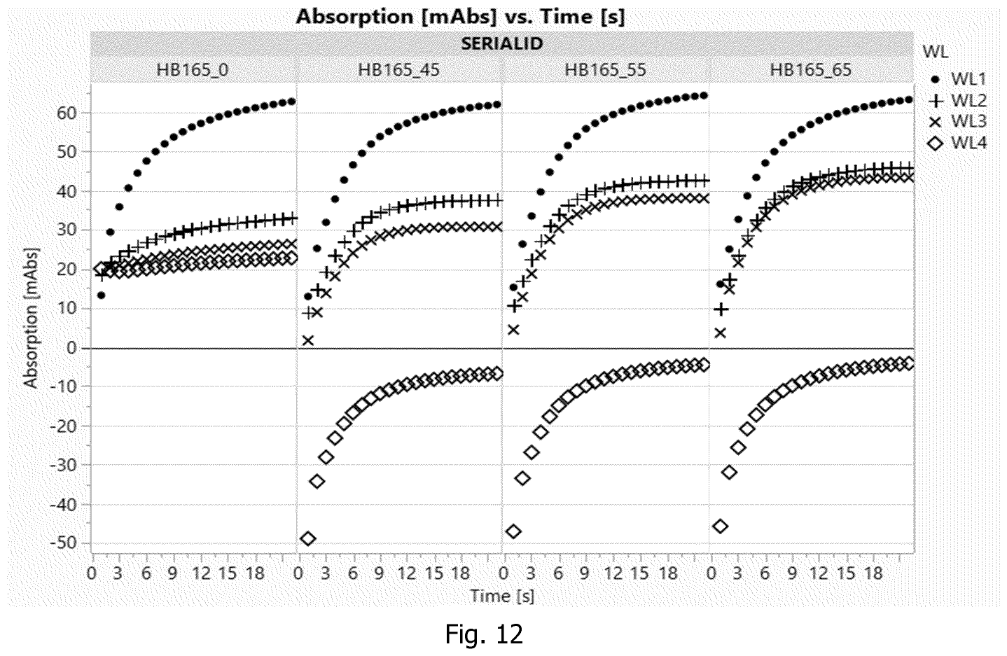

FIG. 12 shows optically detected (absorbance) signal for a setup being similar to the setup used for obtaining the data presented in FIGS. 8-11, and in particular where the porous unit is being directly in contact with the liquid (i.e., no one or more layers present). All scales are linear. Horizontal axes show time in seconds. Vertical axes show optical (absorbance) signal. All curves show temporal development (diffusion), and the sub-graphs show data for, from left to right, liquids with hematocrit (Hct) levels of respectively 0, 45, 55 and 65%. In each sub-graph, four curves (or sets of markers effectively drawing up four curves) are shown, each corresponding to a different wavelength (although the same four wavelengths (WL1, WL2, WL3 and WL4) are employed in each sub-graph).

The porous unit and the setup is or could be similar to the porous mirror and setup described with reference to FIGS. 8-11, where changes with respect to that setup in particular include that there is no one or more layers at the front side of the porous unit (and in particular that there is no reflective, metallic, palladium layer), such as the porous unit being directly in contact with the liquid.

In comparison to a porous mirror (such as a the translucent element with a reflective, metallic, palladium layer at the front side), the porous unit (such as the porous unit being directly in contact with the liquid (i.e., no one or more layers present)) values of intensity, sensitivity and calibration sensitivity have been measured and are provided in the TABLE below.

TABLE

| | | Porous | Porous |
|---|---|---|---|
| Parameter | Optimal | unit | mirror |
| Intensity [counts × 1000] | Highest number | 1123 | 503 |
| Equivalent optical pathlength [μm] | Highest number | 51.5 | 22.8 |
| Calibration sensitivity ratio [%] | 100 | 90.5 | 82.0 |

As seen in the TABLE, each of intensity, sensitivity and calibration sensitivity are improved in the porous unit with respect to the porous mirror.

The intensity increase may be due to a reflectivity due to internal reflection actually being higher relative to reflection due to a metallic, palladium layer (which may, e.g., have merely 60% reflection at 400 nm).

The equivalent optical pathlength is a measure indicating how long a pathlength would have been for a normal cuvette.

The calibration sensitivity ratio is the ratio between the pathlengths of light observed at 415 nm and 450 nm. In a true cuvette the calibration sensitivity ratio would be 100%, and any deviation from 100% is a measure of how “skewed” the system is optically.

Although the present invention has been described in connection with the specified embodiments, it should not be construed as being in any way limited to the presented examples. The scope of the present invention is set out by the accompanying claim set. In the context of the claims, the terms “comprising” or “comprises” do not exclude other possible elements or steps. Also, the mentioning of references such as “a” or “an” etc. should not be construed as excluding a plurality. The use of reference signs in the claims with respect to elements indicated in the figures shall also not be construed as limiting the scope of the invention. Furthermore, individual features mentioned in different claims, may possibly be advantageously combined, and the mentioning of these features in different claims does not exclude that a combination of features is not possible and advantageous.

1. A porous unit for detection of an analyte in a liquid by optical probing, comprising:
a translucent element with a front side, and a backside facing away from the front side, wherein the front side is adapted for being;
contacted directly with the liquid, or
separated from the liquid by one or more layers at the front side of the translucent element, the one or more layers being adapted to:
be non-reflective to light reaching the one or more layers at least at one angle of incidence from the translucent element, and/or
allow internal reflection at an interface of light reaching the interface from the translucent element,
wherein the translucent element comprises pores, wherein the pores are dead end pores extending from respective openings fluidically connecting them with the liquid at the front side into the translucent element,
wherein a cross-sectional dimension of the openings is dimensioned so as to prevent larger particles or debris from entering the pores, while allowing the analyte in the liquid to enter the pores via diffusion.

2. The porous unit according to claim 1, wherein the cross-sectional dimension of the openings of the pores is 1 μm or less, and/or wherein a length of the pores in an axial direction along the pores is less than 100 μm.

3. The porous unit according to claim 1, wherein
a porosity of a given volume of the translucent element comprising pores is between 50% and 5% by volume, and/or
an equivalent pore volume depth is less than 20 μm, wherein the equivalent pore volume depth is defined as a total volume of the pores divided by a front side area over which the openings of the pores are distributed.

4. The porous unit according to claim 1, wherein an inner wall surface of the pores is coated with a hydrophilic coating.

5. The porous unit according to claim 1, wherein the front of the translucent element is separated from the liquid by the one or more layers at the front side of the translucent element, the one or more layers being adapted to be translucent to light reaching the front side at normal incidence from the translucent element.

6. The porous unit according to claim 1, wherein the front of the translucent element is separated from the liquid by the one or more layers at the front side of the translucent element, the one or more layers being adapted to be absorbent to light reaching the front side at normal incidence from the translucent element.

7. The porous unit according to claim 1, wherein the translucent element and/or the one or more layers separating, the front side of the translucent element from the liquid is arranged for enabling internal reflection at the interface between on one side the translucent element and/or one or more layers and on the other side the liquid.

8. The porous unit according to claim 1, wherein the translucent element is provided with reflective elements arranged inside the pores, in a mouth portion thereof, adjacent to the opening at the front side of the translucent element.

9. The porous unit according to claim 8, wherein the reflective elements are provided as a reflective coating covering only a fraction of the circumference of the mouth portion of the pores in the vicinity of the opening, wherein the fraction is about 70% or less.

10. The porous unit according to claim 1, wherein the translucent element comprises, a material which has an attenuation coefficient so that a transmission coefficient of light through the material is at least 50% for a length through the material of 100 micrometers.

11. The porous unit according to claim 1, wherein non-reflective to light entails that at least at the one angle of incidence a reflection coefficient is less than 0.95.

12. The porous unit according to claim 1, further comprising an optical assembly comprising a light guide core, the light guide core comprising an input branch, an output branch, and a coupling interface arranged to contact the backside of the translucent element opposite to the front side, such as wherein the input branch and the output branch are arranged in a common light guide plane arranged perpendicular to a front side surface of the translucent element.

13. The porous unit according to claim 1, further comprising a housing penetrated by a flow channel defining an axial direction, the flow channel comprising a sample space and being arranged so that the porous unit with the front side defining a sensor surface for contacting the liquid faces towards the sample space.

14. A system comprising the porous unit according to claim 1, and further comprising:
one or more light sources, wherein the one or more light sources is adapted to illuminate at least the pores in the translucent element, and/or
a light detector, wherein the light detector is arranged to receive light emerging from the pores in response to an illumination by the one or more light sources, and wherein the light detector is adapted to generate a signal representative of the received light.

15. A system for analyzing a liquid comprising:
a liquid chamber with inlet and outlet ports for feeding and discharging the liquid,
a first detector adapted to provide a first signal representative of a level of an analyte in the liquid, and
one or more further detectors, each further detector being adapted to provide a respective further signal representative of the analyte of the liquid,
wherein the first and further detectors are operable to obtain the first and the one or more of the further signals from the same liquid,
wherein the first detector is configured as a porous unit for the optical detection of the analyte according to claim 1.

16. The system according to claim 14, wherein the system is arranged for optically probing the liquid disposed inside the pores from a side of the front side facing the back side.

17. The system according to claim 14, and comprising both the one or more light sources, and at least the light detector, and wherein each of the one or more light sources and the light detector is placed on a side of the front side facing the back side.

18. The system according to claim 14, wherein
the one or more light sources is adapted to illuminate at least the pores in the translucent element, from a side of the front side facing the back side, and
the light detector is arranged to receive the light emerging from the pores, such as emitted in response to the illumination by the one or more light sources, and wherein the light detector is adapted to generate the signal representative of the received light, which has been emitted from the pores in a direction away from the front side in a direction facing the back side.

19. The system according to claim 14, wherein
the one or more light sources is adapted to illuminate at least the pores in the translucent element, wherein light from the one or more light sources reaching the pores need not have traversed a volume being fluidically connected with the pores and being outside of the translucent element, and
the light detector is arranged to receive the light emerging from the pores, such as emitted in response to the illumination by the one or more light sources, and wherein the light detector is adapted to generate the signal representative of the received light, wherein the light emitted from the pores and reaching the light detector need not have traversed the volume being fluidically connected with the pores and being outside of the translucent element.

20. The system according to claim 14, wherein the system is configured for measuring absorbance.

21. A method for optically detecting an analyte in a liquid comprising:
providing the porous unit according to claim 1;
contacting the front side of the porous unit with the liquid;
optically probing the liquid disposed inside the pores from a side of the front side facing the back side; and
based on the result of the optical probing, establishing an analyte concentration of the liquid.

22. The method for optically detecting an analyte in a liquid according to claim 21, wherein the analyte is
cell-free hemoglobin,
bilirubin, and/or
total protein content.

23. The method for optically detecting an analyte in a liquid according to claim 21, wherein the liquid is a whole blood sample or wherein the liquid is a plasma phase of a whole blood sample.

24. The method for optically detecting an analyte in a liquid according to claim 21, further comprising:
contacting the porous unit with a reference liquid so as to fill the pores by diffusion with the reference liquid, and/or
waiting for a diffusion time to allow for diffusion of the analyte in the liquid into the pores to stabilize.
